(19) United States
(12) Patent Application Publication
(10) Pub. No.: US 2018/0090029 A1
(43) Pub. Date: Mar. 29, 2018

(54) METHOD AND SYSTEM FOR MEDICAL SIMULATION IN AN OPERATING ROOM IN A VIRTUAL REALITY OR AUGMENTED REALITY ENVIRONMENT

(71) Applicant: SIMBIONIX LTD., Airport City (IL)

(72) Inventors: Niv FISHER, Ramat Gan (IL); Lior NESICHI, Rehovot (IL); Eran NEGRIN, Rishon LeZion (IL); Mordechai ZASLAVSKI, Bet-Shemesh (IL); Sophia SLEPNEV, Tel Aviv-Jaffa (IL)

(73) Assignee: SIMBIONIX LTD., Airport City (IL)

(21) Appl. No.: 15/720,143

(22) Filed: Sep. 29, 2017

Related U.S. Application Data
(60) Provisional application No. 62/401,517, filed on Sep. 29, 2016.

(51) Int. Cl. G09B 23/28; G09B 9/00; G06T 19/00; G06Q 50/22; A61B 34/20

(52) U.S. Cl. CPC G09B 23/28; G09B 9/00; G06Q 10/06; G06Q 50/22; A61B 34/20; G06T 19/006

(57) ABSTRACT

Systems and methods for simulating medical procedures in a virtual reality operating room for a training a trainee are provided. A medical procedure can be simulated and a trainee can manually manipulate a medical tool to perform the simulated medical procedure in virtual reality on a virtual reality avatar in the virtual reality simulation.

### CROSS REFERENCE TO RELATED APPLICATION

This patent application claims benefit and priority from co-pending U.S. Provisional Patent Application 62/401,517 filed on Sep. 29, 2016, the entire contents which is hereby incorporated by reference in its entirety.

### FIELD OF THE INVENTION

The invention relates generally to medical simulations used to train medical personnel. In particular, the invention relates to medical simulation in a virtual reality or augmented reality environment that present an operating room experience to a trainee.

### BACKGROUND OF THE INVENTION

Currently, medical simulators can be used to train medical personnel. For example, a trainee (e.g., doctor) can use a computer to perform a computer simulated surgery. The computer simulated surgery can include a display screen that displays images appropriate for a particular surgery, and tools (e.g., haptic tools) that the trainee can manipulate to simulate a surgical experience.

For example, assume a doctor desires to simulate operating on a clogged heart artery of a patient. The doctor can have a set of haptic tools that correspond to the tools that a doctor uses in a real surgery. The doctor selects on a computing device a simulation that corresponds to the clogged heart surgery. The computing device displays on a screen skin of a patient. The doctor can then use one tool of the set of tools to cut the patient open by manually manipulating the haptic tool. The tool can include sensors that senses the doctors manual manipulation, sends the information to the computer simulation and the computer simulation can display the images that correspond to the movements sensed by the tools. Some common tools used for medical simulations include endoscopes, laparoscopes, and/or other operating room machinery.

One difficulty with current simulators is that they typically do not provide a trainee with a realistic experience of being in an operating room. During real operations, there can be many distractions for the surgeon and other medical personnel. For example, a surgeon can be called on an overhead calling system. A nurse can drop a tool being passed to the doctor at a crucial moment. Current medical training simulators can be limited in that they typically do not provide the trainee with a realistic experience.

Therefore, it can be desirable to have a system that can provide a medical trainee with a realistic experience during training.

### SUMMARY OF THE INVENTION

One advantage of the invention can include providing a trainee with a real life experience via performing a medical procedure simulation in a virtual reality or augmented reality operating room. Another advantage of the invention can include an ability to have multiple trainees in the virtual reality (or augmented reality) operating room at the same time, working on the same simulation.

Another advantage of the invention can include an ability to provide the medical simulation view of the procedure on an avatar that is within the virtual reality or augmented reality operating room scene, such that the trainee experiences operating on the avatar. Another advantage of the invention can include an ability to have two trainees in a simulation working remotely on the same simulation.

Another advantage of the invention is that voice commands can be received and incorporated as input into the simulation.

In one aspect, the invention includes a system for simulating medical procedures in a virtual reality operating room for training a trainee. The system can include a user input device for the trainee to select a type of medical procedure to simulate. The system can also include a medical tool including a motion sensor and a touch sensor, the medical tool for the trainee to manually manipulate during the simulation. The system can also include a medical procedure simulation system to receive input from the user input device and the medical tool to execute the simulation of the selected medical procedure. The system can also include a virtual reality simulation system coupled to the medical procedure simulation system to render i) a virtual reality operating room scene that corresponds to the type of medical procedure to simulate, and ii) the simulation of the selected medical procedure into a virtual reality scene. The system can also include a virtual reality headset coupled to the virtual reality simulation system for the trainee to view the virtual reality scene.

In some embodiments, the system includes a connection module that transmits information between the medical procedure simulation system and the virtual reality simulation system such that the virtual reality scene corresponds to the medical procedure simulation output.

In some embodiments, the information transmitted from the virtual reality simulation to the medical procedure simulation comprises information regarding where the trainee is looking inside of the operating room, medical tool animation information, medical procedure information that changes in the virtual reality simulation, and/or any combination thereof.

In some embodiments, the information transmitted from the medical procedure simulation to the virtual reality simulation comprises updates of the medical simulation, the haptic medical tool position, the haptic medical tool orientation, the haptic medical tool type and/or any combination thereof.

In some embodiments, the updates of the medical simulation includes patient behavior, changes that affect vital signs, changes that affect a virtual reality avatar behavior, or any combination thereof. In some embodiments, the system includes a voice activation module that can receive voice commands from the trainee and converts the voice commands into information to be transmitted to the medical simulation.

In some embodiments, the system includes a second user input device for a second trainee to participate in the simulation, a second medical tool for the second trainee to manually manipulate during the simulation, a second medical procedure simulation system coupled to the medical procedure simulation system to: i) receive input from the second user input device and the second medical tool, and ii) to communicate with the medical procedure simulation system to participate in the simulation, a second virtual reality simulation system coupled to the second medical procedure simulation system to render a virtual reality operating room scene that corresponds to the virtual reality operating room scene of the virtual reality simulation system, and a second virtual reality headset coupled to the virtual reality simulation system for the trainee to view the virtual reality scene.

In another aspect, the invention involves a method for simulating medical procedures in a virtual reality operating room for training a trainee. The method involves receiving, via a user input device, a type of medical procedure to simulate. The method also involves receiving, via a medical tool including a motion sensor and a touch sensor, sensed motion and touch of the trainee. The method also involves executing, by a medical procedure simulation system, a simulation of the selected medical procedure based on the received type of medical procedure and the sensed motion and touch of the trainee. The method also involves rendering, by a virtual reality simulation system coupled to the medical procedure simulation system, i) a virtual reality operating room scene that corresponds to the type of medical procedure to simulate, and ii) the simulation of the selected medical procedure into a virtual reality scene. The method also involves displaying, by a virtual reality headset coupled to the virtual reality simulation system, the virtual reality scene.

In some embodiments, the method involves transmitting information between the medical procedure simulation system and the virtual reality simulation system such that the virtual reality scene corresponds to the medical procedure simulation output. In some embodiments, the information transmitted from the virtual reality simulation to the medical procedure simulation comprises information regarding where the trainee is looking inside of the operating room, medical tool animation information, medical procedure information that changes in the virtual reality simulation, and/or any combination thereof.

In some embodiments, the information transmitted from the medical procedure simulation to the virtual reality simulation comprises updates of the medical simulation, the haptic medical tool position, the haptic medical tool orientation, the haptic medical tool type and/or any combination thereof. In some embodiments, the updates of the medical simulation includes patient behavior, changes that affect vital signs, changes that affect a virtual reality avatar behavior, or any combination thereof.

In some embodiments, the method involves receiving voice commands from the trainee and converting the voice commands into information to be used in the medical simulation.

In some embodiments, the method involves receiving, via a second user input device, a request for a second trainee to participate in the simulation, receiving, via a second medical tool, sensed motion and touch information, receiving, via a second medical procedure simulation system, input from the second user input device and the second medical tool, communicating, via a second medical procedure simulation system, with the medical procedure simulation system to participate in the simulation, rendering, via a second virtual reality simulation system, a second virtual reality operating room scene that corresponds to the virtual reality operating room scene of the virtual reality simulation system, and displaying, via a second virtual reality headset, the second virtual reality operating room scene to the second trainee.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure can be used to reference the given feature. Dimensions of features shown in the figures are chosen for convenience and clarity of presentation and are not necessarily shown to scale.

The subject matter regarded as the invention is particularly pointed out and distinctly claimed in the concluding portion of the specification. The invention, however, both as to organization and method of operation, together with objects, features and advantages thereof, can best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of a system for simulating medical procedures in a virtual reality operating room for training a trainee, according to an illustrative embodiment of the invention;

FIG. 2 shows a block diagram of a master and slave system for simulating medical procedures in a virtual reality operating room for training a trainee, according to an illustrative embodiment of the invention;

FIG. 3 shows a flow chart of a method for simulating medical procedures in a virtual reality operating room for training a trainee, according to an illustrative embodiment of the invention; and

FIGS. 4a-4f are diagrams showing examples of a trainee using the simulation system of FIG. 1, 2 or the method of FIG. 3, according to illustrative embodiments of the invention.

It will be appreciated that for simplicity and clarity of illustration, elements shown in the figures have not necessarily been drawn accurately or to scale. For example, the dimensions of some of the elements can be exaggerated relative to other elements for clarity, or several physical components can be included in one functional block or element. Further, where considered appropriate, reference numerals can be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments. For the sake of clarity, discussion of same or similar features or elements cannot be repeated.

Although embodiments of the invention are not limited in this regard, discussions utilizing terms such as, for example, “processing,” “computing,” “calculating,” “determining,” “establishing”, “analyzing”, “checking”, or the like, can refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that can store instructions to perform operations and/or processes. Although embodiments of the invention are not limited in this regard, the terms “plurality” and “a plurality” as used herein can include, for example, “multiple” or “two or more”. The terms “plurality” or “a plurality” can be used throughout the specification to describe two or more components, devices, elements, units, parameters, or the like. The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

In general, a system is provided that can allow for a medical simulator to provide a medical procedure simulation to a trainee in virtual reality. The system can include a medical procedure simulation system that communicates with a virtual reality (VR) simulation system. The trainee (e.g., user) can interact with the medical simulator via one or more medical (e.g., surgical) tools, and experience the medical simulation in virtual reality (or augmented reality). The VR simulation system can use a headset/glasses to present an VR operating room scene to the trainee. The VR operating room scene can include an operating table, vital sign monitors, and/or any equipment that can be present in a real life operating room.

A patient (e.g., avatar or bot) to be operated on can also appear in the operating room scene. The medical procedure simulation system can receive inputs from the medical tools, and as the medical procedure simulation system runs the simulation, the medical procedure simulation information can be used by the VR simulation system to render the medical procedure simulation onto the VR patient. The patient can respond to the trainee's manipulation of the one or more surgical tools during the simulation.

In general, the system can accommodate multiple trainees in one simulation. For example, a surgeon trainee can experience a simulation of a heart surgery simulation on a patient (e.g., avatar) and a nurse trainee can assist the surgeon trainee. The surgeon can see the nurse trainee depicted as a bot within the VR scene, and the nurse trainee can see the surgeon depicted as bot within the VR scene. The surgeon trainee can use haptic tools associated with the medical procedure simulator, and during the simulation, the nurse trainee can pass the surgeon trainee virtual tools in the VR scene.

In some embodiments, an augmented reality (AR) simulation system is used instead of a VR simulation system. In these embodiments, an AR scene is presented to the trainee. The AR scene can include any objects that are typically found in a real-life operating room.

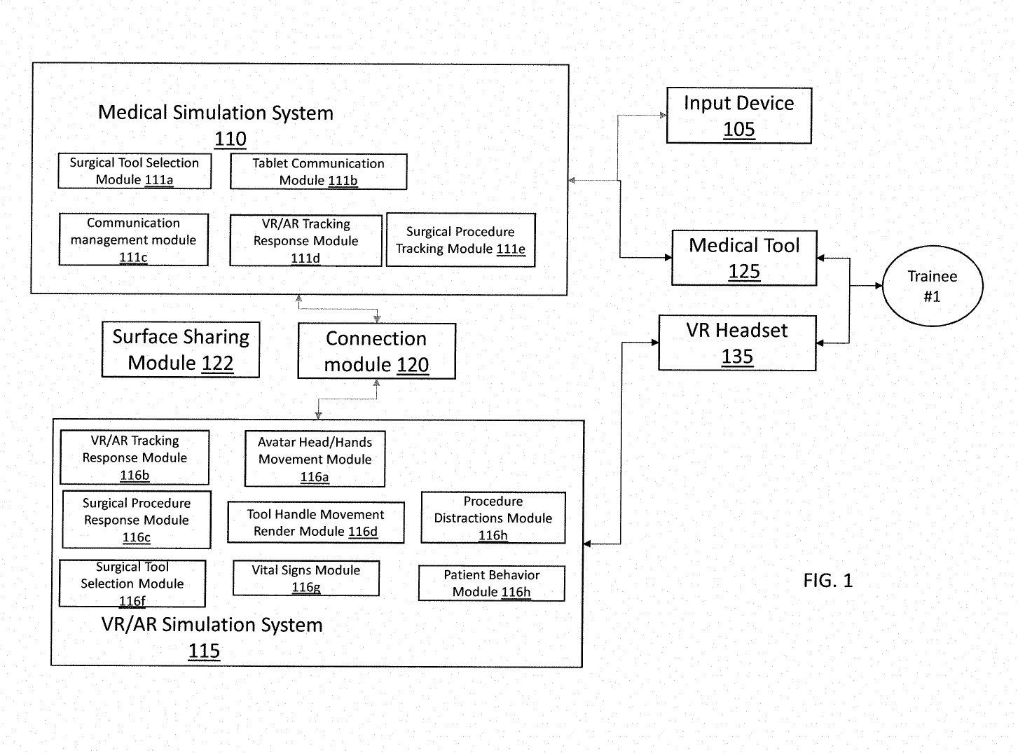

FIG. 1 shows a block diagram of a system for simulating medical procedures in a virtual reality (or augmented reality) operating room for training a trainee, according to an illustrative embodiment of the invention. The system includes an input device 105, a medical procedure simulation system 110, a virtual reality and/or augmented reality (VR/AR) simulation system 115, a connection module 120, a medical tool 125, a virtual reality headset 135. For the purpose of simplicity, the discussion with respect to FIG. 1 will focus on a VR simulation system. However, as is apparent to one of ordinary skill in the art, the simulation system can be virtual reality or augmented reality.

The connection module 120 can include allow for information to flow between the VR simulation system 115 and the medical procedure simulation system 110. For example, inputs received by VR simulation system 115 and/or modifications to the VR operating room scene can be provided to the connection module 120. Inputs received by the medical procedure simulation system 110 and/or medical procedure status information can be provided to the connection module 120. The connection module 120 can be a memory mapped file, one or more pipes, TCP/IP socket communication channels or any combination thereof.

The medical procedure simulation system 110 can be coupled to the input device 105, the medical tool 125 to receive one or more inputs. The medical tool can be a device that can sense motion and touch of the trainee. The medical tool 125 can be a device that is capable of intaking haptic inputs. For example, the medical tool 125 can be a laparoscopic trocar or GI/Bronchoscopy tools. The input device 105 can be a tablet, smart phone, personal computer, touch screen device, or any combination thereof.

The medical procedure simulation system 110 can also be coupled to the VR simulation system 115 via the connection module 120. The medical procedure simulation system 110 can include a central processing unit and/or a graphics processing unit. The medical procedure simulation system 110 can include a two dimensions screen display. The medical procedure simulation system 110 can simulate medical procedures as shown, for example, in U.S. Pat. No. 7,850,456, which is incorporated herein by reference in its entirety.

The medical procedure simulation system 110 can include a surgical tool selection module 111a, a tablet communication module 111b, a communication management module 111c, a virtual reality (VR)/augmented reality (AR) tracking response module 111d, and/or a surgical procedure tracking module 111e.

The tablet communication module 111b can receive input from a trainee. The input can include input that is related to the VR/AR and/or medical simulation. For example, a trainee can select a particular medical procedure to simulate and/or specify a number of participants in the simulation. In some embodiments, a proctor overseeing the training can add to the simulation and receive information from the simulation via the tablet. For example, the proctor can input an injury, and simulation can display to the proctor via the table status (e.g., vessel structure status and/or when the injury is controlled or uncontrolled).

The selected medical procedure can be shared with the VR simulation system 115 such that the VR/AR simulation system can render an operating room environment that corresponds to the selected medical procedure.

The surgical tool selection module 111a can determine one or more surgical tools (e.g., haptic tools or virtual tools) that can be used in the medical procedure simulation based on the selected medical procedure. The one or more surgical tools can be virtual or haptic tools. The surgical tool selection module 111a can also determine which surgical tools can be available in the simulation based on potential tool entry points on the avatar being operated on in the simulation. For example, tool entry points of trocars, open incisions, and/or body cavities. For example, an arterial point of entry for a stent or catheter, can indicate that a laparoscopic trocar can be available. In another example, for an ultrasound simulation an ultrasound probe can be made available. The surgical tool selection module 111a can include a surgical stool status of one or more surgical tools in the medical simulation. For example, a surgical tool status of whether any tool is currently inside/outside of the patient body, whether the particular tool was selected for a particular entry point, a position of the tool, an orientation of the tool and/or properties of the tool (e.g., type and/or name). The surgical tool selection module 111a can also receive surgical tools status information from the VR/AR simulation system 1115. For example, a changed of surgical tool during the medical procedure where the change is from a haptic tool to a virtual tool.

The VR/AR tracking response module 111d can modify the medical simulation based on head movements of the trainee as sensed by the virtual reality headset 135. For example, if a user gazes at one user interface element in the VR scene for longer than 5 seconds, the gaze information can be sent to the medical simulation system. In other examples, if the quantity of anesthetics is changed on the monitoring system in the VR, or if the energy-level for an electro-cautery tool changes before applying it to the tissue, the medical simulation system can be sent this information such that the simulation can be modified.

In some embodiments, the trainee can be wearing VR/AR glove(s) (not shown) that can sense hand motions of the trainee. In these embodiments, the VR/AR tracking response module 111d can modify the medical simulation based on the sensed movement of the gloves.

The surgical procedure tracking module 111e can track a status of the medical procedure simulation and can provide surgical procedure status to be reported to the VR/AR simulation system 115. Surgical procedure status can include changes to the patient (e.g., avatar) during the medical procedure simulation. For example, if the trainee has inserted a tool in a way that causes the avatar's body (e.g., patient's body) to react (e.g., move, bleed and/or shiver), vital signs changes of the avatar, movement of the abdomen with response to the movement of the fetus inside such that an ultrasound view is changed, and/or energy tool can malfunction in mid-surgery such that a message is displayed in the VR scene.

The communication management module 111c can transmit information from the modules shown in the medical procedure simulation system 110 to the connection module 120. The communication management module 111c can transmit the information as soon as its available or with a frequency.

In some embodiments, the medical procedure simulation system 110 include a voice recognition component to receive voice input from the user. For example, if a trainee states “select scalpel” the medical procedure simulation system 110 can receive that audio input, recognize the content of the audio input (e.g., via voice recognition techniques as are known in the art), and the medical procedure simulation system 110 can update the tool in current use as the left trocar entry location and/or remove the previous tool from the simulation. The VR/AR nurse avatar can repeat the tool name and location in its own voice, and the nurse avatar can be displayed to the trainee as obtaining the proper tool and bringing it to the trainee's VR hand or proper location on the patient (e.g., avatar's) body.

The VR simulation system 115 can be coupled to the virtual reality headset 135. The virtual reality headset can be virtual reality headsets as are known in the art. For example, the virtual reality headset can be an Oculus Rift, HTC Vive, or Samsung Gear VR. As is apparent to one of ordinary skill in the art, for embodiments, where the VR simulation system 115 only includes AR, the virtual reality headset 135 can be a AR headset only (e.g., AR glasses). For example, a Microsoft Hololens, or any AR reality headset as is known in the art.

The VR simulation system 115 can include an avatar head/hands movement module 116a, a VR/AR tracking response module 116b, a surgical procedure response module 116c, a tool handle movement render module 116d, a procedure distractions module 116e, a surgical tool selection module 116f, a vital signs module 116g, a patient behavior module 116h, or any combination thereof.

The VR/AR tracking response module 116b can cause the VR scene to respond to the head movements of the trainee as sensed by the virtual reality headset 135. For example, if the trainee turns their head to the left, the VR scene can show the left side of the operating room. If the trainee bends down towards the avatar (e.g., the patient) to, for example, see an incision on the patient more clearly, the VR scene can show the incision zoomed similar to what is experienced by a person in real life.

The surgical procedure response module 116c can receive surgical procedure status information from the medical procedure simulation system 111 (e.g., via the surgical procedure tracking module). The surgical procedure response module 116c can cause the VR scene to be modified according to the surgical procedure status. The surgical procedure response module 116c can include surgical status that is effected by the VR operating room scene. For example, if a second trainee knocks over a table onto an open wound of the avatar.

The surgical tool selection module 116f can receive surgical tool status information from the medical procedure simulation system 110. The surgical tool status information can include rate of insertion into the body or tool orientation. The surgical tool status information can be any information that is related to the tool when in use. The surgical tool status information can be information that related to a particular tool. For example, a laparoscopic stapler/clipper can include a cartridge with a stapling state/clip counter indicator which can be updated when the tool is fired.

The VR simulation system 115 can modify the VR/AR operating room scene based on the surgical tool status information. For example, the VR/AR simulation can render the surgical tool in the VR/AR scene at a location that correlates to the position of the surgical tool in the medical procedure simulation 110. The surgical tool selection module 116f can send status of virtual surgical tools to the medical procedure simulation system 110.

The procedure distractions module 116e can randomly activate distractions that can alter a trainee's behavior. For example, a surgeon trainee can be paged in the VR operating room scene, the OR door can open and staff member can pose a question to a bot on the operating team, some of the staff (e.g., bots or other participants) can start chatting and/or the nurse can provide a tool other than what was indicated.

The vital signs module 116g can modify the VR operating room scene based on vital sign information from the medical procedure simulation system 110. For example, the VR operating room scene can include one or more vital sign monitors which can display the vital sign information (e.g., pulse, temperature and/or oxygen level). The avatar's behavior can correspond to the vital signs. For example, in the case of an injury to a large vessel a sudden decrease in blood pressure can be displayed.

The patient behavior module 116h can modify the avatars visual appearance based on the surgical procedure status from the medical procedure simulation system 110. For example, the VR avatar can appear as bleeding, having palpitations and/or stomach deflation.

In some embodiments, a second trainee can participate in the simulation via a second system. In these embodiments, the VR/AR simulation system 110 can receive inputs and/or output information to the second system. The tool handle movement render module 116d can receive tool information from the second system and determine what tool information to display in the VR operating room scene.

The avatar head/hands movement module 116a can receive head and/or hand movement information from a second system and render that movement in the VR scene for the trainee of the first system.

The trainee and/or avatar within the VR/AR scene can be medical personnel, including nurses, doctors, physicians assistants, medical personnel related to certain procedures (e.g., a hip replacement manufacturer doctor that monitors hip replacement surgeries).

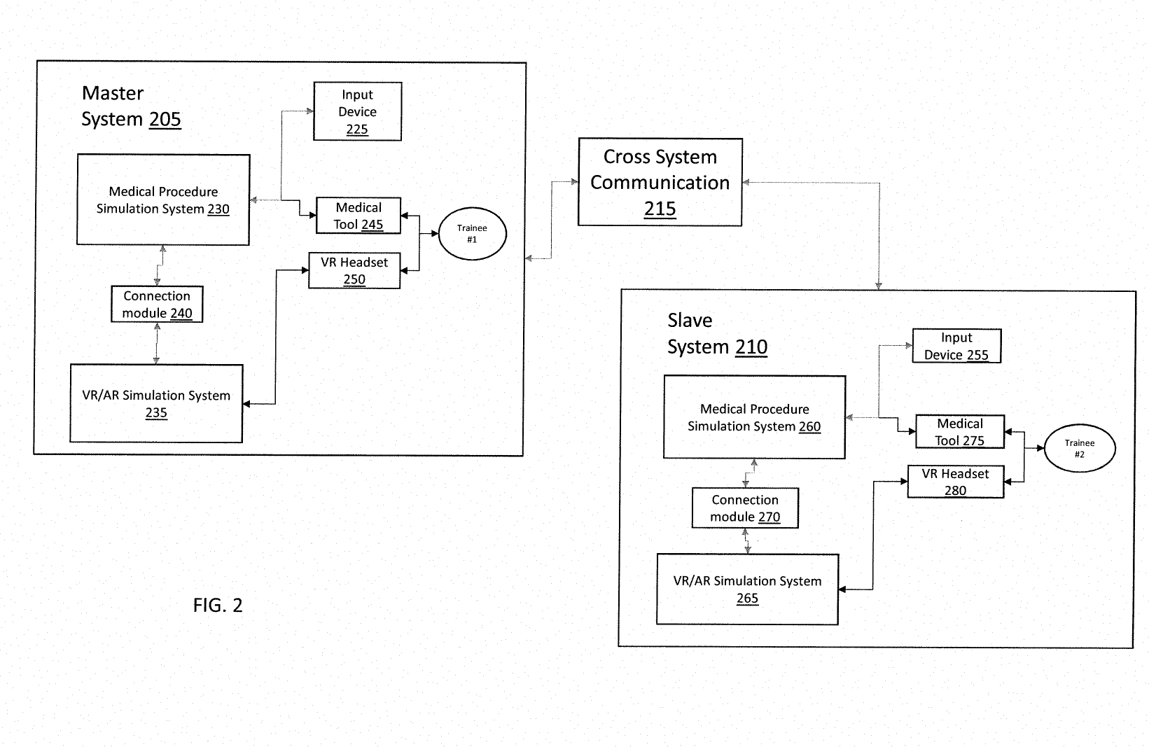

FIG. 2 shows a block diagram of a master and slave system for simulating medical procedures in a virtual reality operating room for training a trainee, according to an illustrative embodiment of the invention. The system can include a master system 205, a slave system 210, and a cross system communication module 215.

The cross-system communication module 215 can allow information to flow between the master system 205 and the slave system 210. The cross-system communication module 215 can be a memory mapped file, a pipe, a TCP/IP socket.

The master system 205 can include an input device 225, a medical procedure simulation system 230, a virtual reality and/or augmented reality (VR/AR) simulation system 235, a connection module 240, a medical tool 245, and/or a virtual reality headset 250. The slave system 210 can include an input device 255, a medical procedure simulation system 260, a virtual reality and/or augmented reality (VR/AR) simulation system 265, a connection module 270, a medical tool 275, and/or a virtual reality headset 280. The master system 205 and/or the slave system 220 modules can operate in the same manner as described above with respect to FIG. 1.

During operation, a first trainee can run a simulation on the master system 205. The master system 205 can initiate a server and can wait for a connection from the slave system 210. A second trainee can run a simulation on the slave system 210. The second trainee can specify on the slave system 210 that it is to run in slave mode. The cross-system communication module 215 can initiate communication between the master system 205 and the slave system 210. The master system 205 an transmit medical operation simulation status and VR/AR simulation status the slave system 210. For example, voice inputs, tool movement inputs, VR/AR tracking movements. The slave system 210 can transmit medical operation simulation status and VR/AR simulation status to the master system 205. The slave system 210 and the master system 205 can receive respective inputs and update their respective medical operation simulation systems and VR/AR simulation systems with the cross-system information.

The cross-system communication module 215 can be a memory mapped file that can share information between two processes even if, for example, they are in different terminal sessions.

In some embodiments, multiple slave simulation systems connect to a master simulation system. In this manner, 3, 4 and/or any number of trainees can participate in a given procedure simulation.

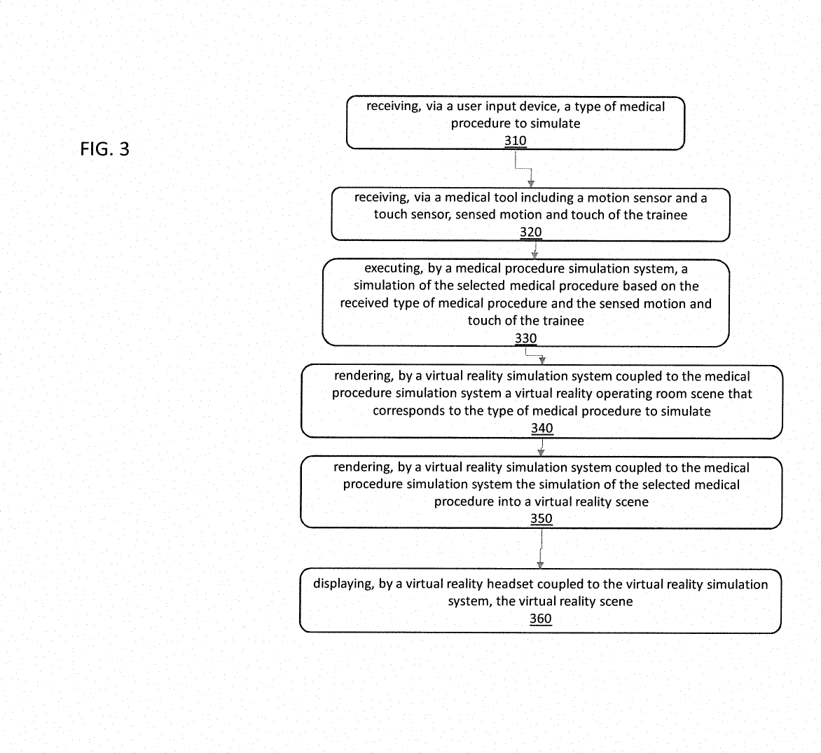

FIG. 3 shows a flow chart of a method for simulating medical procedures in a virtual reality operating room for training a trainee. The method involves receiving (e.g., via the input device 105 as described above in FIG. 1) a type of medical procedure to simulate (Step 310). The type of medical procedure can be specified by a trainee, a person who wants to monitor the trainee (e.g., teacher) or any other user. The type of medical procedure can be a surgery, diagnostic procedures using ultrasound and/or other imaging modalities, anesthesia, cardiovascular interventions, and/or emergency room treatment.

The surgery can be on an infant, child and/or adult. In some embodiments, the surgery is on an animal. In some embodiments, once the medical procedure is specified, a virtual reality scene or augmented reality scene that corresponds to the medical procedure specified is rendered and presented to the trainee by a VR/AR headset.

The method can also involve receiving (e.g., via the medical tool 125 as described above in FIG. 1) sensed motion and touch of the trainee (Step 320). The trainee can be wearing VR/AR gloves or holding a tool having one or more sensors for sensing touch and motion.

The method can also involve executing a simulation of the selected medical procedure (e.g., via the medical procedure simulation system 110, as described above in FIG. 1) based on the received type of medical procedure and the sensed motion and touch of the trainee (Step 330). As the trainee moves the tool, the simulation can receive the location and sensor information from the tool. The simulation can interpret the movement and touch and modify the simulation output based on that movement and touch. For example, a trainee can be operating on a simulated heart. If the trainee moves the tool slowly near an artery as shown in the VR/AR headset, the simulation can interpret that movement as causing a slow cut in the heart. The simulation can cause the heart to bleed or appear open depending on the location and the touch.

The method can also involve rendering (e.g., via the VR/AR simulation system 115) a virtual reality operating room scene that corresponds to the type of medical procedure to simulate (Step 340). The virtual reality operating room can be rendered at a rate between 90 and 120 frames per second.

The method can also involve rendering (e.g., via the VR/AR simulation system 115) the simulation of the selected medical procedure into a virtual reality scene (Step 350). The simulation can be rendered onto an avatar in the VR/AR scene. The avatar can correspond to the type of procedure (e.g., child's bypass surgery).

The method can also involve displaying (e.g., via the virtual reality headset 135) the virtual reality scene (Step 360). In some embodiments, the method includes displaying via a AR headset a AR scene.

In some embodiments, the trainee selects an assisted procedure to simulate. For example, during a simulation of a medical procedure on an VR/AR avatar, the trainee using the VR/AR headset and a haptic tool, can point the haptic tool at a body part of the VR/AR avatar. A second avatar (e.g., a nurse bot and/or an anesthesiologist bot) in the VR scene can instruct the user (e.g., tell the user how to minimize injury due to a mistake).

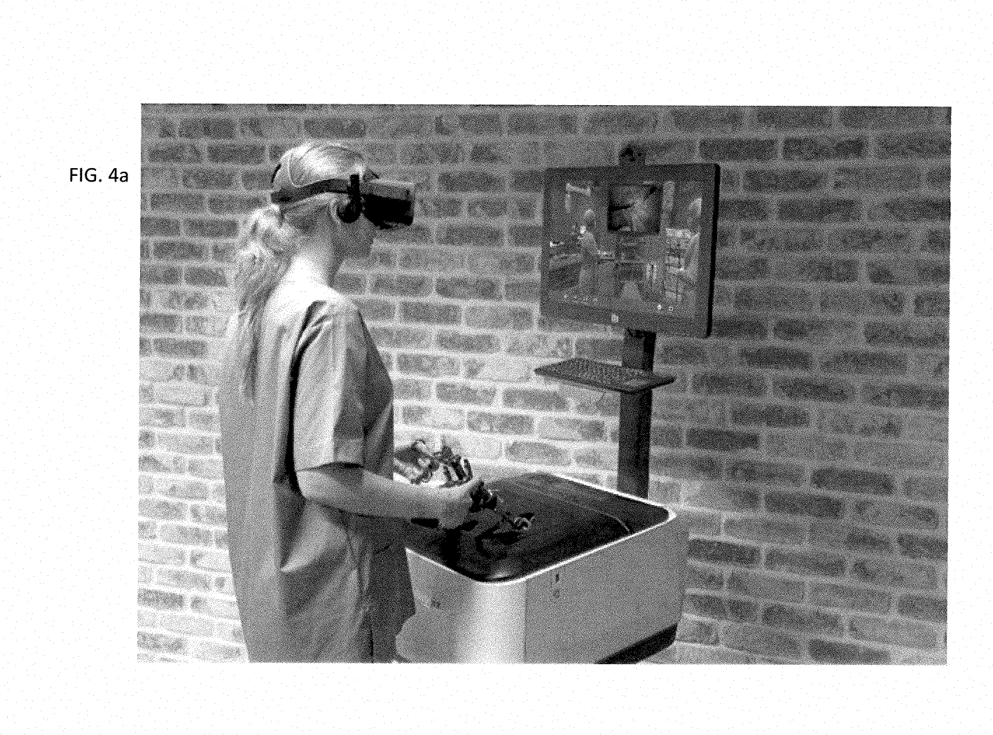

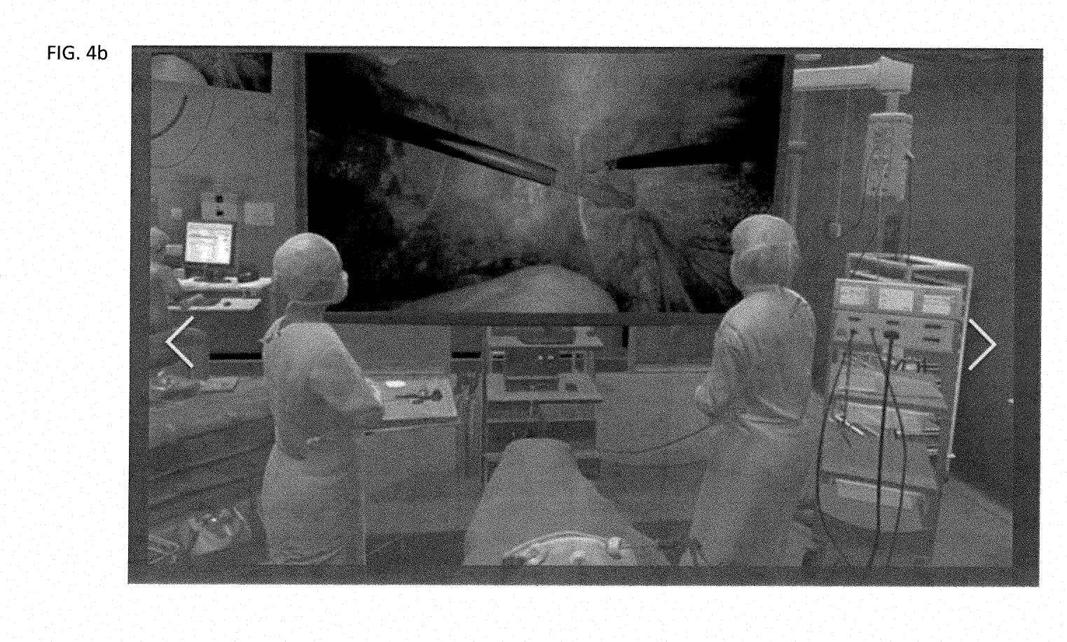

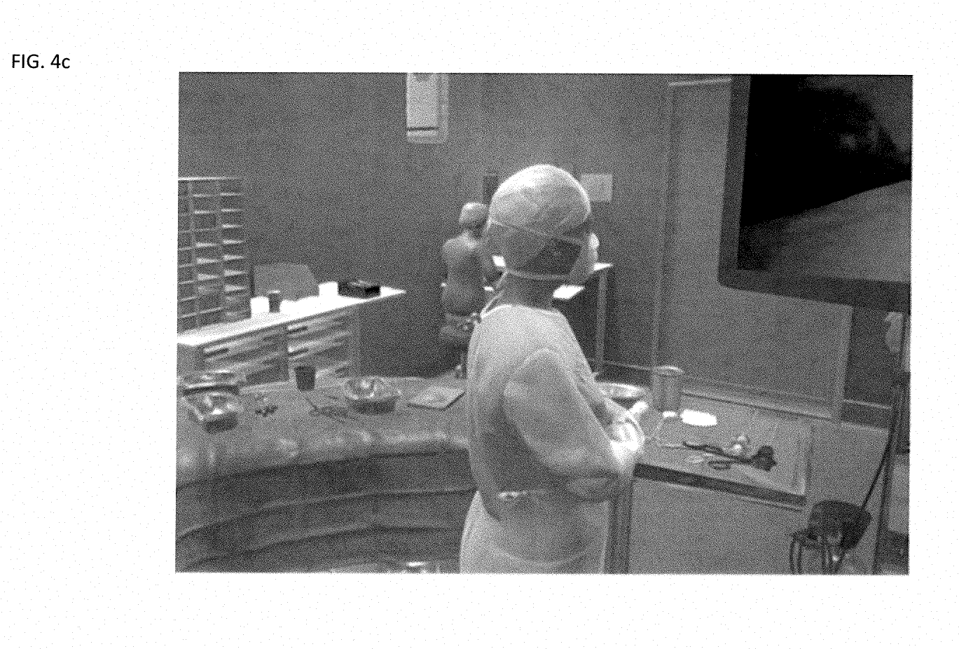

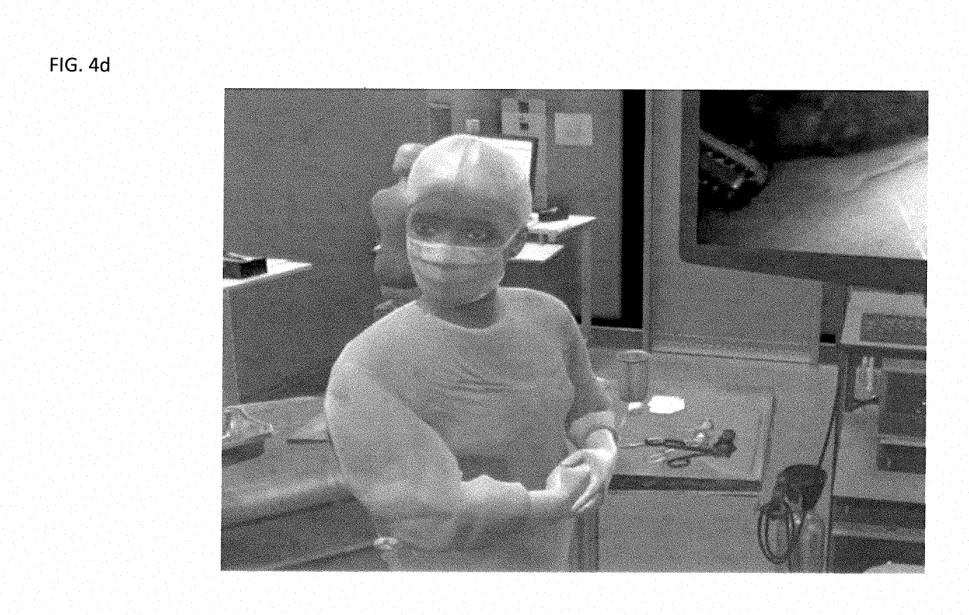

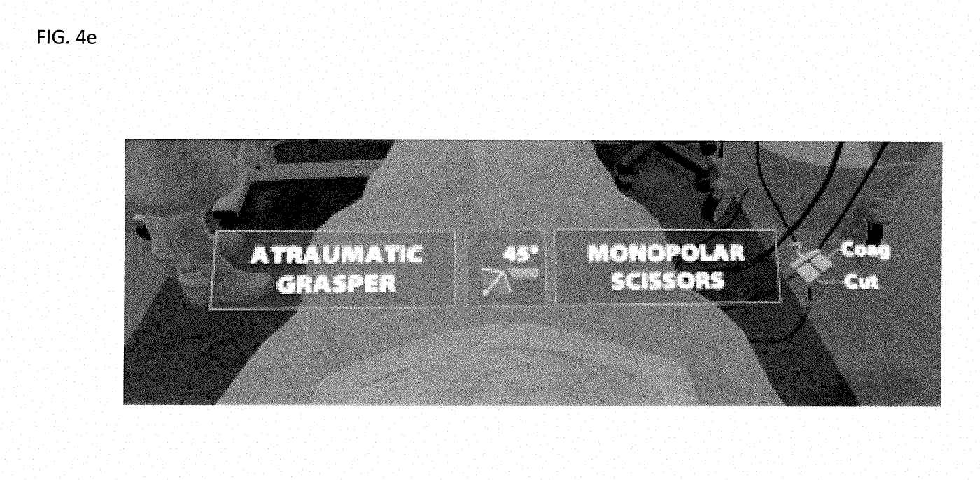

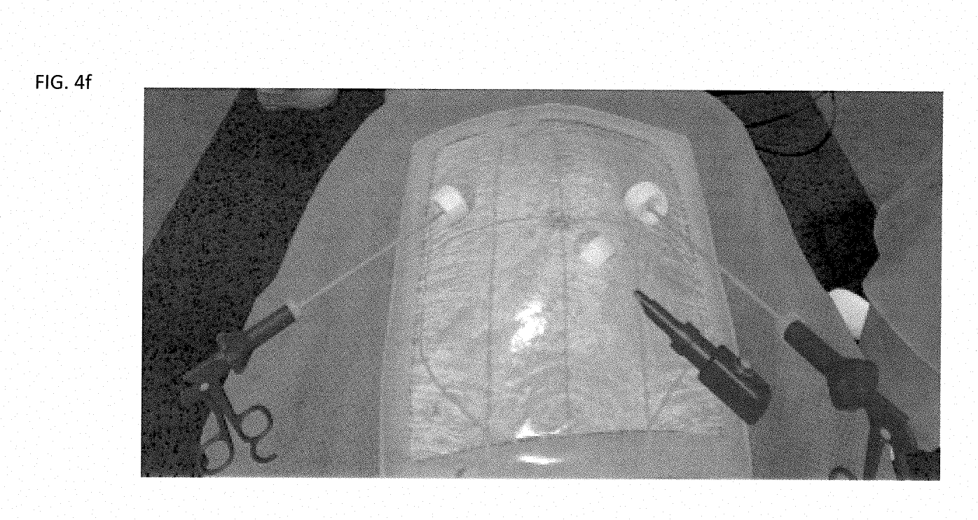

FIGS. 4a-4f are diagrams showing examples of a trainee using the simulation system of FIG. 1, 2 or the method of FIG. 3, according to illustrative embodiments of the invention. FIG. 4a shows an example of trainee holding two medical tools wearing a VR headset. Also shown is a two-dimensional screen showing a two-dimensional view of simulation. FIG. 4b shows an example of a two-dimensional screen shot of a virtual reality scene as viewed by the trainee in the virtual reality headset. The medical procedure simulation is shown on a screen, with two bots in the operating room. FIG. 4c shows an example of a screen shot of a virtual reality scene with a nurse bot in the operating room. FIG. 4d shows an example of a screen shot of a virtual reality scene with a nurse bot in the operating room talking to the trainee. FIG. 4e shows a screen shot of a user interface superimposed on the virtual reality scene. FIG. 4f shows an example of a screen shot a virtual reality scene with multiple tools for the trainee to use in virtual reality.

The above-described methods can be implemented in digital electronic circuitry, in computer hardware, firmware, and/or software. The implementation can be as a computer program product (e.g., a computer program tangibly embodied in an information carrier). The implementation can, for example, be in a machine-readable storage device for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by an apparatus and can be implemented as special purpose logic circuitry. The circuitry can, for example, be a FPGA (field programmable gate array) and/or an ASIC (application-specific integrated circuit). Modules, subroutines, and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device, a transmitting device, and/or a computing device. The display device can be, for example, a cathode ray tube (CRT) and/or a liquid crystal display (LCD) monitor. The interaction with a user can be, for example, a display of information to the user and a keyboard and a pointing device (e.g., a mouse or a trackball) by which the user can provide input to the computer (e.g., interact with a user interface element). Other kinds of devices can be used to provide for interaction with a user. Other devices can be, for example, feedback provided to the user in any form of sensory feedback (e.g., visual feedback, auditory feedback, or tactile feedback). Input from the user can be, for example, received in any form, including acoustic, speech, and/or tactile input.

The computing device can include, for example, a computer, a computer with a browser device, a telephone, an IP phone, a mobile device (e.g., cellular phone, personal digital assistant (PDA) device, laptop computer, electronic mail device), and/or other communication devices. The computing device can be, for example, one or more computer servers. The computer servers can be, for example, part of a server farm. The browser device includes, for example, a computer (e.g., desktop computer, laptop computer, and tablet) with a World Wide Web browser (e.g., Microsoft® Internet Explorer® available from Microsoft Corporation, Chrome available from Google, Mozilla® Firefox available from Mozilla Corporation, Safari available from Apple). The mobile computing device includes, for example, a personal digital assistant (PDA).

Website and/or web pages can be provided, for example, through a network (e.g., Internet) using a web server. The web server can be, for example, a computer with a server module (e.g., Microsoft® Internet Information Services available from Microsoft Corporation, Apache Web Server available from Apache Software Foundation, Apache Tomcat Web Server available from Apache Software Foundation).

The storage module can be, for example, a random access memory (RAM) module, a read only memory (ROM) module, a computer hard drive, a memory card (e.g., universal serial bus (USB) flash drive, a secure digital (SD) flash card), a floppy disk, and/or any other data storage device. Information stored on a storage module can be maintained, for example, in a database (e.g., relational database system, flat database system) and/or any other logical information storage mechanism.

The above-described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

The above described networks can be implemented in a packet-based network, a circuit-based network, and/or a combination of a packet-based network and a circuit-based network. Packet-based networks can include, for example, the Internet, a carrier internet protocol (IP) network (e.g., local area network (LAN), wide area network (WAN), campus area network (CAN), metropolitan area network (MAN), home area network (HAN), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, Bluetooth®, code-division multiple access (CDMA) network, time division multiple access (TDMA) network, global system for mobile communications (GSM) network), and/or other circuit-based networks.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the foregoing detailed description, numerous specific details are set forth in order to provide an understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments.

1. A system for simulating medical procedures in a virtual reality operating room for training a trainee, the system comprising:
a user input device for the trainee to select a type of medical procedure to simulate;
a medical tool including a motion sensor and a touch sensor, the medical tool for the trainee to manually manipulate during the simulation;
a medical procedure simulation system to receive input from the user input device and the medical tool to execute the simulation of the selected medical procedure;
a virtual reality simulation system coupled to the medical procedure simulation system to render i) a virtual reality operating room scene that corresponds to the type of medical procedure to simulate, and ii) the simulation of the selected medical procedure into a virtual reality scene; and
a virtual reality headset coupled to the virtual reality simulation system for the trainee to view the virtual reality scene.

2. The system of claim 1 further comprising:
a connection module that transmits information between the medical procedure simulation system and the virtual reality simulation system such that the virtual reality scene corresponds to the medical procedure simulation output.

3. The system of claim 2 wherein the information transmitted from the virtual reality simulation to the medical procedure simulation comprises information regarding where the trainee is looking inside of the operating room, medical tool animation information, medical procedure information that changes in the virtual reality simulation, and/or any combination thereof.

4. The system of claim 2 wherein the information transmitted from the medical procedure simulation to the virtual reality simulation comprises updates of the medical simulation, the haptic medical tool position, the haptic medical tool orientation, the haptic medical tool type and/or any combination thereof.

5. The system of claim 4 wherein the updates of the medical simulation includes patient behavior, changes that affect vital signs, changes that affect a virtual reality avatar behavior, or any combination thereof.

6. The system of claim 1 further comprising a voice activation module that can receive voice commands from the trainee and converts the voice commands into information to be transmitted to the medical simulation.

7. The system of claim 1 wherein the system further comprises:
a second user input device for a second trainee to participate in the simulation;
a second medical tool for the second trainee to manually manipulate during the simulation;
a second medical procedure simulation system coupled to the medical procedure simulation system to: i) receive input from the second user input device and the second medical tool, and ii) to communicate with the medical procedure simulation system to participate in the simulation;
a second virtual reality simulation system coupled to the second medical procedure simulation system to render a virtual reality operating room scene that corresponds to the virtual reality operating room scene of the virtual reality simulation system; and
a second virtual reality headset coupled to the virtual reality simulation system for the trainee to view the virtual reality scene.

8. A method for simulating medical procedures in a virtual reality operating room for training a trainee, the method comprising:
receiving, via a user input device, a type of medical procedure to simulate;
receiving, via a medical tool including a motion sensor and a touch sensor, sensed motion and touch of the trainee;
executing, by a medical procedure simulation system, a simulation of the selected medical procedure based on the received type of medical procedure and the sensed motion and touch of the trainee;
rendering, by a virtual reality simulation system coupled to the medical procedure simulation system, i) a virtual reality operating room scene that corresponds to the type of medical procedure to simulate, and ii) the simulation of the selected medical procedure into a virtual reality scene;
displaying, by a virtual reality headset coupled to the virtual reality simulation system, the virtual reality scene.

9. The method of claim 8 further comprising:
transmitting information between the medical procedure simulation system and the virtual reality simulation system such that the virtual reality scene corresponds to the medical procedure simulation output.

10. The method of claim 9 wherein the information transmitted from the virtual reality simulation to the medical procedure simulation comprises information regarding where the trainee is looking inside of the operating room, medical tool animation information, medical procedure information that changes in the virtual reality simulation, and/or any combination thereof.

11. The method of claim 9 wherein the information transmitted from the medical procedure simulation to the virtual reality simulation comprises updates of the medical simulation, the haptic medical tool position, the haptic medical tool orientation, the haptic medical tool type and/or any combination thereof.

12. The method of claim 11 wherein the updates of the medical simulation includes patient behavior, changes that affect vital signs, changes that affect a virtual reality avatar behavior, or any combination thereof.

13. The method of claim 8 further comprising receiving voice commands from the trainee and converting the voice commands into information to be used in the medical simulation.

14. The method of claim 1 wherein the method further comprises:
receiving, via a second user input device, a request for a second trainee to participate in the simulation;
receiving, via a second medical tool, sensed motion and touch information;
receiving, via a second medical procedure simulation system, input from the second user input device and the second medical tool;
communicating, via a second medical procedure simulation system, with the medical procedure simulation system to participate in the simulation;
rendering, via a second virtual reality simulation system, a second virtual reality operating room scene that corresponds to the virtual reality operating room scene of the virtual reality simulation system; and
displaying, via a second virtual reality headset, the second virtual reality operating room scene to the second trainee.
